(19) United States
(12) Patent Application Publication
(10) Pub. No.: US 2022/0000430 A1
(43) Pub. Date: Jan. 6, 2022

(54) DETERMINATION APPARATUS, SENSOR APPARATUS, DETERMINATION METHOD, AND NON-TRANSITORY COMPUTER-READABLE RECORDING MEDIUM

(71) Applicant: NEC Corporation, Tokyo (JP)

(72) Inventors: Kenichiro FUKUSHI, Tokyo (JP); Chenhui HUANG, Tokyo (JP); Yusuke SEKIGUCHI, Sendai-shi (JP); Keita HONDA, Sendai-shi (JP); Haruki YAGUCHI, Sendai-shi (JP); Shinichi IZUMI, Sendai-shi (JP); Dai OWAKI, Sendai-shi (JP)

(73) Assignee: NEC Corporation, Tokyo (JP)

(21) Appl. No.: 17/362,449

(22) Filed: Jun. 29, 2021

(30) Foreign Application Priority Data
Jul. 3, 2020 (JP) 2020-115938

(51) Int. Cl. A61B 5/00; G01P 3/44; G01P 15/18; A61B 5/11; G16H 50/30

(52) U.S. Cl. CPC A61B 5/7275; G01P 3/44; A61B 5/6898; A61B 5/112; G16H 50/30; G01P 15/18

(57) ABSTRACT

A determination apparatus acquires a feature amount of a whipping motion of a foot and determines a gait disorder risk on the basis of the feature amount.

### CROSS-REFERENCE TO RELATED APPLICATION

This application is based upon and claims the benefit of priority from Japanese Patent Application No. 2020-115938 filed Jul. 3, 2020, the disclosure of which is incorporated in its entirety by reference.

### TECHNICAL FIELD

The present invention relates to a determination apparatus, a sensor apparatus, a determination method, and a non-transitory computer-readable recording medium.

### BACKGROUND ART

It is known that walking analysis is performed for the purpose of preventing the exacerbation of knee disorders. For example, Japanese Unexamined Patent Application, First Publication No. 2017-202236 discloses a walking analysis technology aimed at preventing pathogenesis and/or exacerbation of a knee disorder of a subject.

### SUMMARY

It is known that a whipping motion during walking has a possibility of being associated with various risk factors that cause a gait disorder. “Whipping motion” refers to motion in which an ankle makes an adduction or abduction around a leg axis in a swing phase or the like of a leg in walking. This whipping motion is associated with a gait disorder due to a pain, restriction of a range of motion of a joint, and neurological disorders. As an example, rotational motion in a whipping motion can be a stress factor applied to a knee, can cause a shearing force on a knee joint, and can be a risk factor or an exacerbating factor for arthropathy. Therefore, it is required to be able to determine a gait disorder risk due to such a whipping motion.

Therefore, an example object of the present invention is to provide a determination apparatus, a sensor apparatus, a determination method, and a non-transitory computer-readable recording medium that solve the above-described problems.

A first example aspect of the present invention is a determination apparatus including: at least one memory configured to store instructions; and at least one processor configured to execute the instructions to: acquire a feature amount of a whipping motion of a foot; and determine a gait disorder risk on the basis of the feature amount.

A second example aspect of the present invention is a sensor apparatus provided in a shoe sole, and the sensor apparatus includes: at least one memory configured to store instructions; and at least one processor configured to execute the instructions to: measure an acceleration or an angular velocity of a foot; and calculate a feature amount of a whipping motion of the foot on the basis of the acceleration or the angular velocity.

A third example aspect of the present invention is a determination method including: acquiring a feature amount of a whipping motion of a foot; and determining a gait disorder risk on the basis of the feature amount.

According to the present invention, a gait disorder risk due to a whipping motion can be determined.

### BRIEF DESCRIPTION OF THE DRAWINGS

FIG. 1 is a diagram showing a schematic configuration of a gait disorder risk determination system according to a first example embodiment of the present invention.

FIG. 2 is a diagram showing a hardware configuration of a determination apparatus, a first sensor, and a second sensor according to the first example embodiment of the present invention.

FIG. 3 is a functional block diagram of the determination apparatus, the first sensor, and the second sensor according to the first example embodiment of the present invention.

FIG. 4 is a first diagram illustrating a whipping motion according to the first example embodiment of the present invention.

FIGS. 5A and 5B are second diagrams illustrating a whipping motion according to the first example embodiment of the present invention.

FIG. 6 is a third diagram illustrating a whipping motion according to the first example embodiment of the present invention.

FIG. 7 is a fourth diagram illustrating a whipping motion according to the first example embodiment of the present invention.

FIG. 8 is a diagram illustrating an example of usage of the determination apparatus, the first sensor, and the second sensor according to the first example embodiment of the present invention.

FIG. 9 is a diagram showing a processing flow of each apparatus in the gait disorder risk determination system according to the first example embodiment of the present invention.

FIG. 10 is a diagram showing a processing flow of the determination apparatus according to the first example embodiment of the present invention.

FIG. 11 is a diagram showing an example of a threshold used by a risk determination unit according to the first example embodiment of the present invention.

FIG. 12 is a functional block diagram of a determination apparatus according to a second example embodiment of the present invention.

FIG. 13 is a diagram showing a schematic configuration of a gait disorder risk determination system according to a fifth example embodiment of the present invention.

FIG. 14 is a diagram showing a minimum configuration of a determination apparatus according to an example embodiment of the present invention.

FIG. 15 is a diagram showing a processing flow of the determination apparatus with the minimum configuration according to the example embodiment of the present invention.

### DETAILED DESCRIPTION OF THE INVENTION

Hereinafter, a gait disorder risk determination apparatus according to an example embodiment of the present invention will be described with reference to the drawings.

### First Example Embodiment

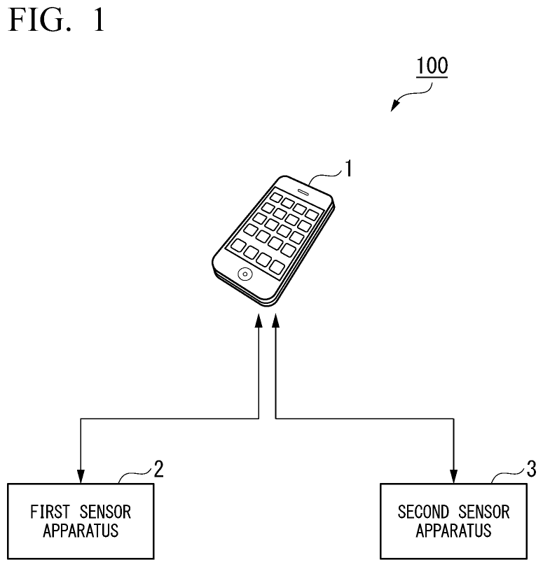

FIG. 1 is a diagram showing a schematic configuration of a gait disorder risk determination system according to a first example embodiment of the present invention.

As shown in FIG. 1, a gait disorder risk determination system 100 is configured by at least a determination apparatus 1, a first sensor apparatus 2, and a second sensor apparatus 3. The determination apparatus 1 connects to the first sensor apparatus 2 and the second sensor apparatus 3 and communicates with the first sensor apparatus 2 and the second sensor apparatus 3 to acquire sensing information detected by sensors of the first sensor apparatus 2 and the second sensor apparatus 3.

The first sensor apparatus 2 and the second sensor apparatus 3 are each attached to a shoe sole, measure an acceleration or an angular velocity of a foot, and calculate a feature amount of a whipping motion of the foot on the basis of the acceleration or the angular velocity. As an example, the first sensor apparatus 2 calculates a feature amount of a whipping motion of a left foot, and the second sensor apparatus 3 calculates a feature amount of a whipping motion of the right foot. The determination apparatus 1 receives the feature amounts of the whipping motions from the first sensor apparatus 2 and the second sensor apparatus 3 and determines a gait disorder risk on the basis of the feature amounts. It should be noted that the present example embodiment will describe an example in which both the first sensor apparatus 2 and the second sensor apparatus 3 are used to determine a gait disorder on the basis of feature amounts of the whipping motions of both the left foot and the right foot. However, only one of the first sensor apparatus 2 and the second sensor apparatus 3 may be used to determine a gait disorder on the basis of a feature amount of the whipping motion of either the left foot or the right foot.

The determination apparatus 1 may be a mobile terminal such as a smartphone. Moreover, the determination apparatus 1 may be any apparatus as long as it receives feature amounts of whipping motions from the first sensor apparatus 2 and the second sensor apparatus 3 and performs processing of determining a gait disorder risk. For example, the determination apparatus 1 may be a server apparatus provided remotely.

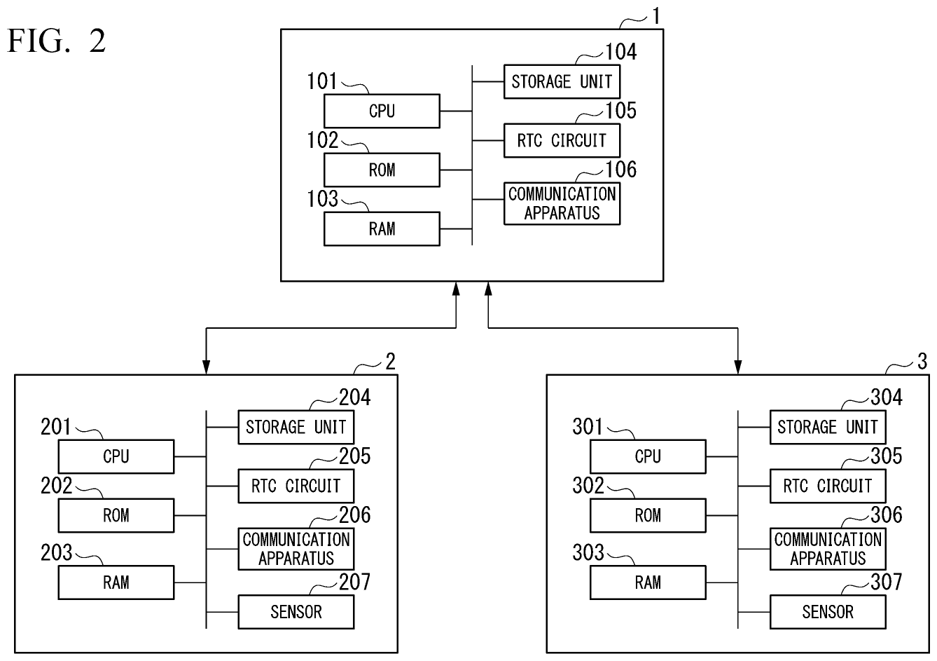

FIG. 2 is a diagram showing a hardware configuration of a determination apparatus, a first sensor, and a second sensor.

The determination apparatus 1 is a computer that includes hardware such as a central processing unit (CPU) 101, a read only memory (ROM) 102, a random access memory (RAM) 103, a storage unit 104, a real time clock (RTC) circuit 105, and a communication apparatus 106.

Moreover, the first sensor apparatus 2 is a computer that includes hardware such as a CPU 201, a ROM 202, a RAM 203, a storage unit 204, an RTC circuit 205, a communication apparatus 206, and a sensor 207.

Moreover, the second sensor apparatus 3 is a computer that includes hardware such as a CPU 301, a ROM 302, a RAM 303, a storage unit 304, an RTC circuit 305, a communication apparatus 306, and a sensor 307.

In the present example embodiment, the sensor 207 of the first sensor apparatus 2 and the sensor 307 of the second sensor apparatus 3 are each configured by an inertial measurement unit (IMU) that senses an acceleration and/or an angular velocity based on a motion of a foot when a user walks.

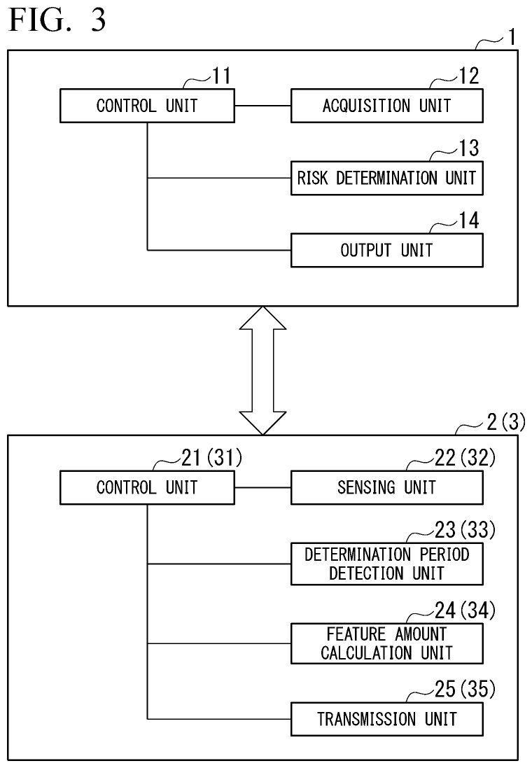

FIG. 3 is a functional block diagram of the determination apparatus, the first sensor, and the second sensor.

The determination apparatus 1 executes a gait disorder risk determination program stored in advance. Thereby, the determination apparatus 1 exerts the functions of at least a control unit 11, an acquisition unit 12, a risk determination unit 13, and an output unit 14.

The control unit 11 of the determination apparatus 1 controls other functional units of the determination apparatus 1.

The acquisition unit 12 of the determination apparatus 1 acquires a feature amount of a whipping motion of a foot.

The risk determination unit 13 of the determination apparatus 1 determines a gait disorder risk of the user on the basis of the feature amount of the whipping motion of the foot.

The output unit 14 of the determination apparatus 1 outputs information such as a determination result by the risk determination unit 13 to an output destination.

Moreover, the first sensor apparatus 2 executes a sensing program stored in advance. Thereby, the first sensor apparatus 2 exerts the functions of at least a control unit 21, a sensing unit 22, a determination period detection unit 23, a feature amount calculation unit 24, and a transmission unit 25.

The control unit 21 of the first sensor apparatus 2 controls other functional units of the first sensor apparatus 2. The sensing unit 22 of the first sensor apparatus 2 acquires an acceleration and/or an angular velocity based on a motion of the left foot when the user walks from the sensor 207 such as an IMU. The determination period detection unit 23 of the first sensor apparatus 2 detects a whipping motion determination period of the left foot on the basis of the acceleration and/or the angular velocity of the left foot. The feature amount calculation unit 24 of the first sensor apparatus 2 calculates a feature amount of a whipping motion of the left foot on the basis of the acceleration and/or the angular velocity of the left foot. The transmission unit 25 of the first sensor apparatus 2 transmits the feature amount of the whipping motion of the left foot to the determination apparatus 1.

Moreover, the second sensor apparatus 3 executes a sensing program stored in advance. Thereby, the second sensor apparatus 3 exerts the functions of at least a control unit 31, a sensing unit 32, a determination period detection unit 33, a feature amount calculation unit 34, and a transmission unit 35.

The control unit 31 of the second sensor apparatus 3 controls other functional units of the second sensor apparatus 3. The sensing unit 32 of the second sensor apparatus 3 acquires an acceleration and/or an angular velocity based on a motion of the right foot when the user walks from the sensor 307 such as an IMU. The determination period detection unit 33 of the second sensor apparatus 3 detects a whipping motion determination period of the right foot on the basis of the acceleration and/or the angular velocity of the right foot. The feature amount calculation unit 34 of the second sensor apparatus 3 calculates a feature amount of a whipping motion of the right foot on the basis of the acceleration and/or the angular velocity of the right foot. The transmission unit 35 of the second sensor apparatus 3 transmits the feature amount of the whipping motion of the right foot to the determination apparatus 1.

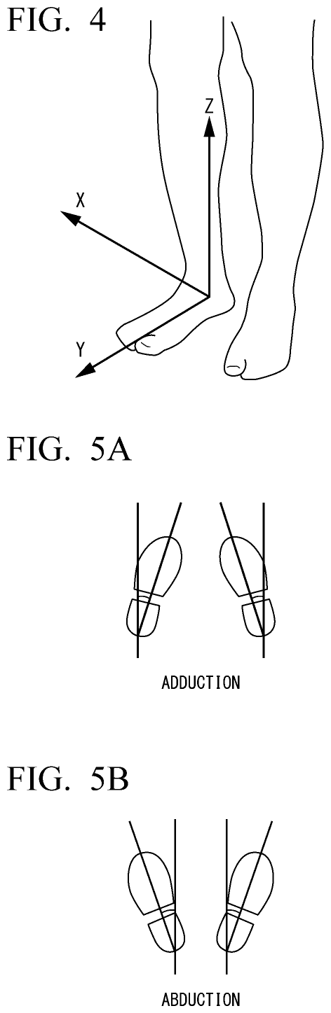

FIG. 4 is a first diagram illustrating a whipping motion.

In the present example embodiment, the determination apparatus 1 acquires the feature amount indicating a rotation angle around a leg axis below a knee from each of the first sensor apparatus 2 and the second sensor apparatus 3 provided on soles of shoes attached to the feet. The leg axis corresponds to a Z-axis in FIG. 4. When a leg and a sole of a foot are at a right angle with a vicinity of an ankle as an origin, an axis in a direction from the origin to a toe is a Y-axis, an axis perpendicular to the Y-axis and the Z-axis is an X-axis, a rotation angle around the X axis is regarded as a pitch angle, a rotation angle around the Y axis is regarded as a roll angle, and a rotation angle around the Z axis is regarded as a yaw angle. The determination apparatus 1 determines a gait disorder risk on the basis of the magnitude of the yaw angle in a period from a pre-swing to an end time of an initial swing in a motion cycle of each foot in walking.

FIGS. 5A and 5B are second diagrams illustrating a whipping motion.

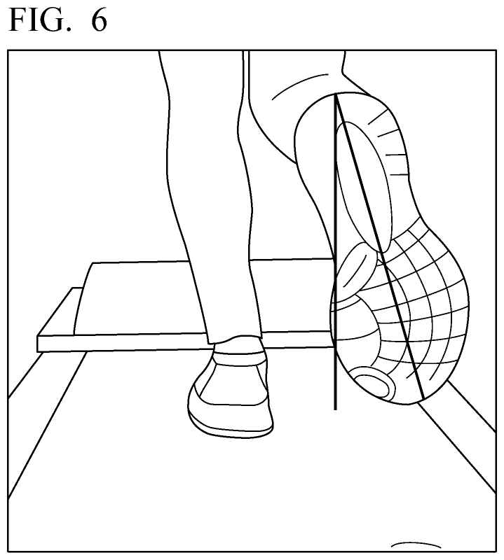

FIG. 6 is a third diagram illustrating a whipping motion.

For example, in a case of the right foot, generation of the yaw angle in a clockwise direction with respect to a position in which the foot is facing in a forward movement direction is called abduction (FIG. 5B). Moreover, generation of the yaw angle in a counterclockwise direction with respect to the position in which the foot is facing in the forward movement direction is called adduction (FIG. 5A). When the angle of abduction or adduction is large during a period from a pre-swing to an end time of an initial swing in a motion cycle of each foot in walking, it can cause a stress on a knee, can cause a shearing force on a knee joint, and can be a risk factor or exacerbating factor for arthropathy. FIG. 6 illustrates a state in which abduction occurs during walking.

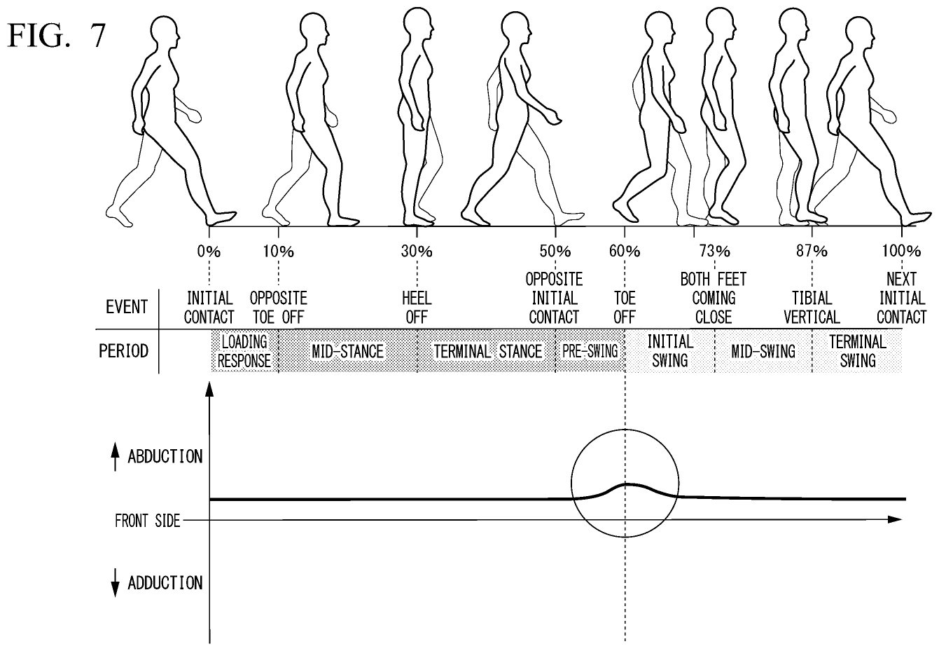

FIG. 7 is a fourth diagram showing a whipping motion.

FIG. 7 shows a temporal relationship between walking motion of a person (upper row), the names of divided periods in a motion cycle in the walking motion (middle row), and rotation angles with respect to a leg axis below a knee in the motion cycle (lower row). As shown in the upper row of FIG. 7, in the walking motion of the person, one cycle of a motion cycle of the walking motion is defined as a period from landing of the right foot to the next landing of the right foot. The heel of the right foot lands at 0% in an elapsed time in one cycle of the motion cycle of the walking motion (initial contact). In that case, the toes of the left foot, which are on a contralateral side, are separated from the ground at around 10% in the elapsed time thereafter (opposite toe off). Thereafter, the heel of the left foot starts to be lifted at around 30% in the elapsed time (heel off). Thereafter, the heel of the left foot, which is on the contralateral side, contacts the ground at around 50% in the elapsed time (opposite initial contact). Thereafter, the toes of the right foot are separated from the ground at around 60% in the elapsed time (toe off). Thereafter, the left and right feet come close to each other at around 73% in the elapsed time before the right foot strides forward (both feet coming close). Thereafter, a tibial of the right foot becomes vertical at around 87% in the elapsed time (tibial vertical). Thereafter, the heel of the right foot contacts the ground, and one cycle of the motion cycle in the next walking motion starts at 100% in the elapsed time.

In such a motion cycle, a period of around 0% to 10% in the elapsed time of one cycle is called a loading response, a period of around 10% to around 30% is called a mid-stance, a period of around 30% to around 50% is called a terminal stance, a period of around 50% to around 60% is called a pre-swing, a period of around 60% to around 73% is called an initial swing, a period of around 73% to around 87% is called a mid-swing, and a period of around 87% to 100% is called a terminal swing. As an example, a whipping motion may occur during a period from the pre-swing to the initial swing. Then, as shown in the lower row of FIG. 7, when a rotation angle in the abduction direction or a rotation angle in the adduction direction is equal to or larger than a threshold during the period from the pre-swing to the initial swing, it can cause a shearing force on a knee joint and can be a risk factor or exacerbating factor for arthropathy. Therefore, the determination apparatus 1 determines a gait disorder risk on the basis of the whipping motion that occurs during such a period from the pre-swing to the initial swing. Hereinafter, the period from the pre-swing to the initial swing will be referred to as a whipping motion determination period.

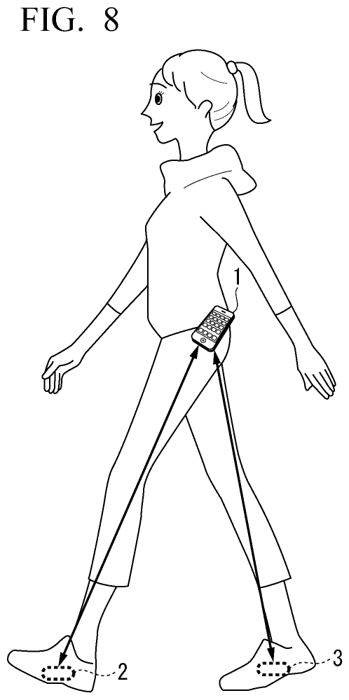

FIG. 8 is a diagram illustrating an example of usage of the determination apparatus, the first sensor, and the second sensor.

As an example, the determination apparatus 1 may be carried by a user. Then, the first sensor apparatus 2 is attached in an insole of a shoe of the left foot and near the arch of the left foot of the user. Moreover, the second sensor apparatus 3 is attached in an insole of a shoe of the right foot and near the arch of the right foot of the user. Then, the first sensor apparatus 2 and the second sensor apparatus 3 calculate feature amounts of the whipping motions on the basis of accelerations and angular velocities detected in accordance with movements of the feet due to walking of the user and transmit the calculated feature amounts to the determination apparatus 1.

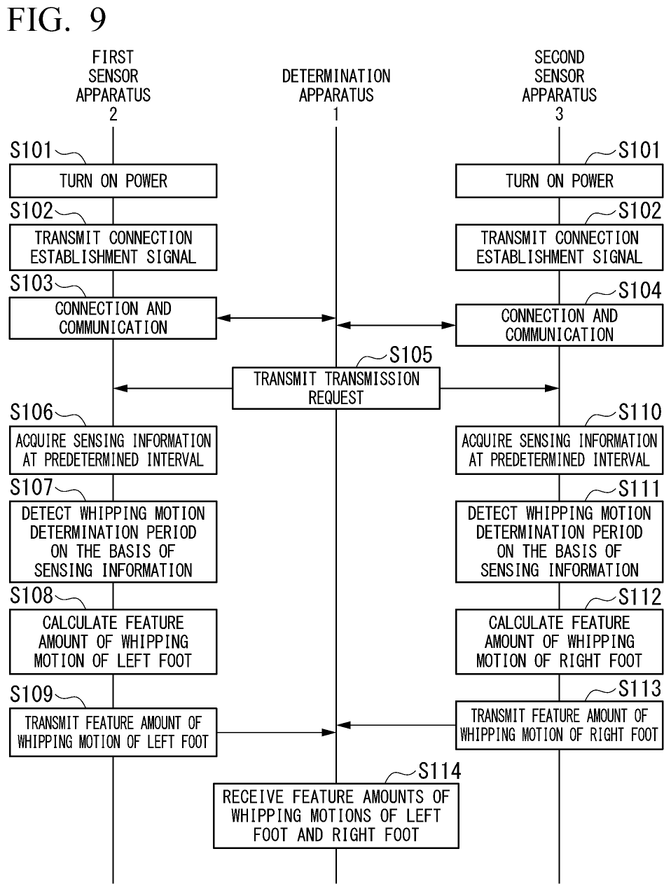

FIG. 9 is a diagram showing a processing flow of each apparatus in the gait disorder risk determination system.

A user turns on power of the first sensor apparatus 2 and the second sensor apparatus 3 (step S101). Thereby, the communication apparatus 206 of the first sensor apparatus 2 and the communication apparatus 306 of the second sensor apparatus 3 transmit connection establishment signals (step S102). These communication apparatuses 206 and 306 have a wireless communication function such as Bluetooth Low Energy (BLE; registered trademark) or Wifi (registered trademark) as an example and use this function to connect to other apparatuses and communicate with the other apparatuses.

The user operates the determination apparatus 1 to allow its connection to the first sensor apparatus 2 and communication with the first sensor apparatus 2. Thereby, the determination apparatus 1 connects to the first sensor apparatus 2 and communicates with the first sensor apparatus 2 (step S103). Similarly, the user operates the determination apparatus 1 to allow its communication with the second sensor apparatus 3 and connection to the second sensor apparatus 3. Thereby, the determination apparatus 1 connects to the second sensor apparatus 3 and communicate with the second sensor apparatus 3 (step S104). The user inputs an instruction to start determination to the determination apparatus 1. Then, the control unit 11 of the determination apparatus 1 transmits a transmission request to the first sensor apparatus 2 and the second sensor apparatus 3 (step S105).

Hereinafter, processing of the first sensor apparatus 2 will be described. The control unit 21 of the first sensor apparatus 2 outputs the transmission request to the sensing unit 22. Then, the sensing unit 22 acquires sensing information such as an acceleration and/or an angular velocity from the sensor 207 at a predetermined interval (step S106). The predetermined interval may be an interval of a short period of time such as, for example, an interval of 1 millisecond. The sensing unit 22 outputs the sensing information to the determination period detection unit 23 and the feature amount calculation unit 24.

The determination period detection unit 23 detects a whipping motion determination period on the basis of the sensing information (step S107). Specifically, the determination period detection unit 23 detects a start time and an end time of a motion cycle of walking motion of the user on the basis of the acceleration indicated by the sensing information. As an example, the determination period detection unit 23 detects a timing at which the heel of the right foot lands using the acceleration, also detects a timing at which the heel of the right foot lands next using the acceleration, and detects those timings of landing of the heel as the start time and the end time of the motion cycle. When the start time of the motion cycle is 0% of the cycle and the end time thereof is 100% of the cycle, the determination period detection unit 23 detects a time when an elapsed time from the start time is 50% to determine the detected time as a start time of the pre-swing, detects a time when an elapsed time from the start time is around 73% to determine the detected time as an end time of the initial swing, and determines a period between these times as the whipping motion determination period. Moreover, the determination period detection unit 23 detects a time when an elapsed time from the start time is 10% to determine the detected time as a start time of the mid-stance and detects a time when an elapsed time from the start time is around 30% to determine the detected time as an end time of the mid-stance. The determination period detection unit 23 outputs the start time and the end time of the whipping motion determination period to the feature amount calculation unit 24 for each motion cycle of the walking motion.

The feature amount calculation unit 24 acquires sensing information from the sensing unit 22 and acquires the start time and the end time of the whipping motion determination period from the determination period detection unit 23. The feature amount calculation unit 24 acquires the sensing information detected during the whipping motion determination period. The feature amount calculation unit 24 calculates a feature amount W of the whipping motion of the left foot in the whipping motion determination period using the sensing information (step S108). The feature amount calculation unit 24 calculates the feature amount W of the whipping motion of the left foot by using, for example, Equations (1) to (4).

Specifically, f(t) indicating the difference between an adduction/abduction angle θ(t) and an adduction/abduction angle θ(T_{MSt}) in the mid-stance is calculated by Equation (1). t_{MSt} indicates a time of the mid-stance.

f(t)=θ(t)−θ(t_{MSt}). (1)

The magnitude w of the whipping motion is defined as the absolute value of the difference between the adduction/abduction angle θ(t) in a predetermined time range t₀ to t₁ and the adduction/abduction angle θ(t_{MSt}) of the mid-stance. The time range t₀ to t₁ is preferably set as, for example, the pre-swing to the initial swing (50% to 73% in one cycle of the motion cycle of the walking motion), which is before and after striding.

$$w = {max}_{{t}_{0} < t < {t}_{1}} ‖ f ( t ) ‖ \qquad ( 2 )$$

Here, tₘₐₓ is set as a time when the magnitude w of the whipping motion is maximum.

$${t}_{max} = {argmax}_{{t}_{0} < t < {t}_{1}} ‖ f ( t ) ‖ \qquad ( 3 )$$

At this time, the sign of f(tₘₐₓ) indicates adduction or abduction of the whipping motion (a lateral whip when the sign is positive and a medial whip when the sign is negative). Using these, the feature amount W of the whipping motion is defined as Equation (4).

$$W = \{ \begin{matrix}+ w & ( f ( {t}_{max} ) > 0 ) \\ - w & ( f ( {t}_{max} ) \leq 0 )\end{matrix} \qquad ( 4 )$$

The sign of W indicates the direction of the whipping motion (a lateral whip when the sign is positive and a medial whip when the sign is negative), and the absolute value of W indicates the magnitude of the whipping motion.

Alternatively, the feature amount calculation unit 24 may calculate the feature amount W of the whipping motion of the left foot by regarding the whipping motion as an integration of a variation amount with the adduction/abduction angle of the mid-stance phase as a reference using Equation (5). The sign of W indicates the direction of the whipping motion (a lateral whip when the sign is positive and a medial whip when the sign is negative), and the absolute value of W indicates the magnitude of the whipping motion.

W=∫ₜ_{₀}^{t}^{₁}{θ(t)−θ(t_{MS})}dt (5)

Then, the feature amount calculation unit 24 outputs the feature amount of the whipping motion of the left foot to the transmission unit 25. The feature amount calculation unit 24 repeats the calculation of the feature amount of the whipping motion of the left foot for each motion cycle of the walking motion, and outputs the calculated feature amounts to the transmission unit 25. Each time the transmission unit 25 acquires the feature amount of the whipping motion of the left foot, the transmission unit 25 repeats to transmit information of the feature amount to the determination apparatus 1 (step S109). The control unit 21 of the first sensor apparatus 2 determines whether to end the processing. For example, when the control unit 21 receives a processing stop request from the determination apparatus 1 or detects that power is off, the control unit 21 ends the processing. Otherwise, the control unit 21 repeats the processing on and after step S107.

The second sensor apparatus 3 also performs the processing similar to that of the first sensor apparatus 2 described above (steps S110 to S113). Thereby, the second sensor apparatus 3 repeats calculation of a feature amount of a whipping motion of the right foot and transmits information of the feature amount to the determination apparatus 1 (step S113). The determination apparatus 1 receives the feature amount of the whipping motion of the left foot from the first sensor apparatus 2 for each motion cycle of the walking motion and receives the feature amount of the whipping motion of the right foot from the second sensor apparatus 3 for each motion cycle of the walking motion (step S114).

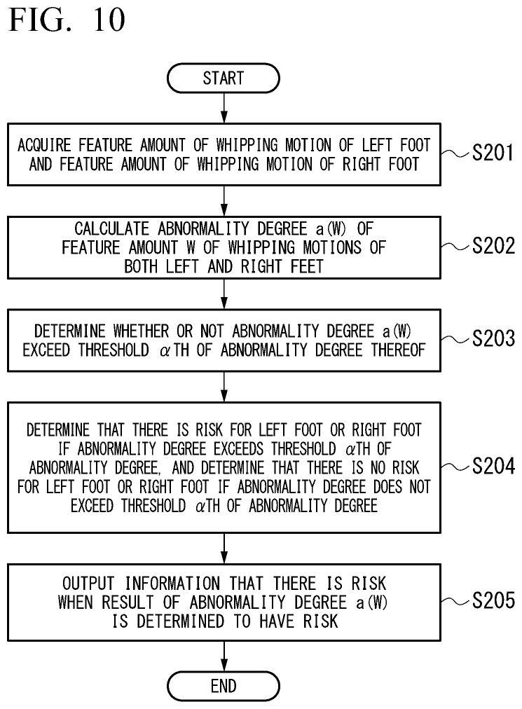

FIG. 10 is a diagram showing a processing flow of the determination apparatus 1.

The acquisition unit 12 of the determination apparatus 1 acquires the received feature amount of the whipping motion of the left foot and the received feature amount of the whipping motion of the right foot (step S201). The acquisition unit 12 outputs the feature amounts of the whipping motions to the risk determination unit 13.

The determination apparatus 1 stores an average value μ and a standard deviation 6 of the feature amounts W of the whipping motions obtained from a normal distribution model, which is a statistical model for determining a gait disorder risk, in the storage unit 104 in advance. This statistical model is a statistical model created from whipping motions of persons who are not recognized as having a gait disorder risk. Whether or not the gait disorder risk is recognized is determined by doctors or physical therapists. The risk determination unit 13 calculates an abnormality degree a(W) of the feature amount W of the whipping motion for each of the both feet using Equation (6) (step S202).

$$a ( W ) = \frac{{( W - μ )}^{2}}{{σ}^{2}} \qquad ( 6 )$$

Then, the risk determination unit 13 determines whether the abnormality degree a(W) of the feature amount W of the whipping motion calculated for each of the both feet exceeds a threshold αth of the abnormality degree (step S203). The risk determination unit 13 determines that there is a risk for the left foot or the right foot if the abnormality degree a(W) of the feature amount W of the whipping motion of the left foot or the right foot exceeds the threshold αth of the abnormality degree, and determines that there is no risk for the left foot or the right foot if the abnormality degree a(W) of the feature amount W of the whipping motion of the left foot or the right foot does not exceed the threshold αth of the abnormality degree (step S204).

The risk determination unit 13 determines whether there is a risk or no risk for the left foot or the right foot as to the abnormality degree a(W) for each motion cycle of the walking motion, and if a predetermined ratio of the determination results of the abnormality degree a(W) for the left foot or the right foot for all the motion cycles generated in a predetermined period during the walking is at risk, the risk determination unit 13 outputs information that there is a risk for the left foot or the right foot to a display apparatus such as, for example, a liquid crystal screen (step S205). For example, when the determination apparatus 1 is a smartphone, the information that there is a risk for the left foot or the right foot is displayed on a liquid crystal screen of the smartphone. Thereby, the user can learn that he/she is performing walking motion having a risk for the left foot or the right foot in his/her own walking motion.

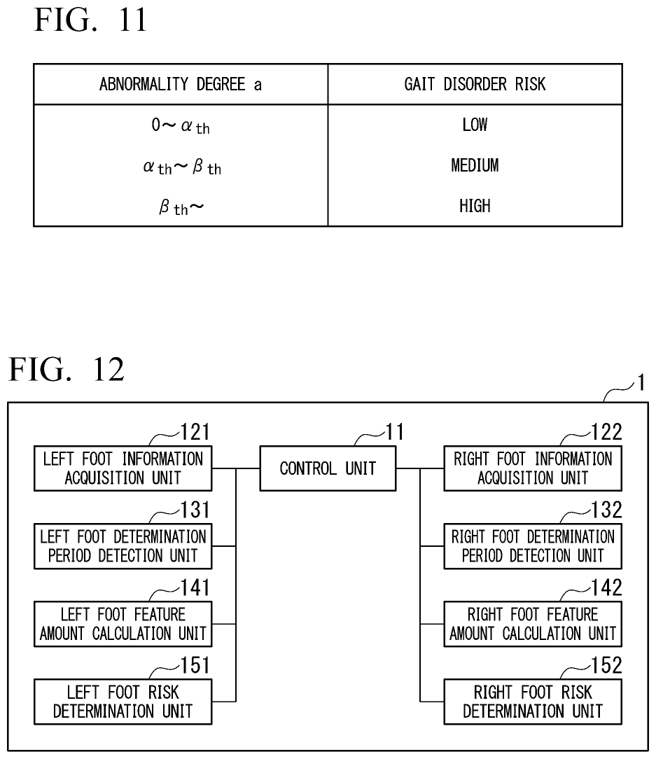

FIG. 11 is a diagram showing an example of a threshold used by the risk determination unit 13.

The risk determination unit 13 may determine the multistage risk of a gait disorder using a plurality of thresholds instead of determining the gait disorder risk with binary values such as with risk or without risk. For example, as shown in FIG. 11, the storage unit 104 stores each range of the abnormality degree a(W) for three stages of the gait disorder risk including “low,” “medium,” and “high.” For example, the storage unit 104 stores information of the gait disorder risk “low” when the abnormality degree a(W) satisfies 0≤a(W)≤αth, stores information of the gait disorder risk “medium” when the abnormality degree a(W) satisfies αth<a(W)<βth, and stores information of the gait disorder risk “high” when the abnormality degree a(W) satisfies βth<a(W), wherein (αth<βth). The risk determination unit 13 determines whether the gait disorder risk for the left foot or the right foot is “low,” “medium,” or “high,” and outputs the determination result to a display apparatus or the like. Thereby, the user can learn what the stage of risk is for the left foot or the right foot in his/her walking motion.

In the above-described processing, the risk determination unit 13 determines the gait disorder risk by using the average value μ and the standard deviation σ of the feature amounts W of the whipping motions based on the normal distribution model created from whipping motions of persons who are not recognized as having a gait disorder risk. Here, the risk determination unit 13 may further determine that there is a risk of a specific disease symptom by using an average value μ and a standard deviation σ of the feature amounts W of the whipping motions based on normal distribution models created from whipping motions for each of persons corresponding to different disease symptoms that cause gait disorders such as gonarthrosis, hallux valgus, or the like.

For example, when it is determined that there is a risk of a gait disorder and when the risk of the gait disorder is determined to be “medium” or “high,” the risk determination unit 13 further compares the average value μ and the standard deviation 6 of the feature amounts W of the whipping motions for each disease symptom with those of the feature amount W of the whipping motion of the user. If the feature amount W of the whipping motion of the user is within the standard deviation 6 of the feature amounts of whipping motions of a certain disease symptom or close to the average value μ thereof, the risk determination unit 13 outputs information indicating that there is a risk of the disease symptom to a display apparatus. Thereby, the determination apparatus 1 can determine that there is a risk of a specific disease symptom on the basis of the whipping motion of the user.

Moreover, the risk determination unit 13 may use different thresholds for a whipping motion in the abduction direction and a whipping motion in the adduction direction for each of the right foot and the left foot to determine whether or not (or stages) there is a gait disorder risk due to the whipping motion in the abduction direction and whether or not (or stages) there is a gait disorder risk due to the whipping motion in the adduction direction. For example, the storage unit 104 of the determination apparatus 1 stores each range of the abnormality degree a(W) for the three stages of the gait disorder risk including “low,” “medium,” and “high” for each of the left foot and the right foot.

Specifically, the storage unit 104 stores information of the gait disorder risk “low” when the abnormality degree a(W) of the whipping motion in the abduction direction for the left foot satisfies 0≤a(W)≤αth1, stores information of the gait disorder risk “medium” when the abnormality degree a(W) for the left foot satisfies αth1<a(W)≤βth1, and stores information of the gait disorder risk “high” when the abnormality degree a(W) for the left foot satisfies βth1<a(W). On the basis of this information, the risk determination unit 13 determines whether the gait disorder risk of the whipping motion of the left foot in the abduction direction is “low,” “medium,” or “high,” and outputs the determination result to a display apparatus or the like. Similarly, the storage unit 104 stores information of the gait disorder risk “low” when the abnormality degree a(W) of the whipping motion in the adduction direction for the left foot satisfies 0≤a(W)≤αth1′, stores information of the gait disorder risk “medium” when the abnormality degree a(W) for the left foot satisfies αth1′<a(W)≤βth1′, and stores information of the gait disorder risk “high” when the abnormality degree a(W) for the left foot satisfies (βth1′<a(W). On the basis of this information, the risk determination unit 13 determines whether the gait disorder risk of the whipping motion of the left foot in the adduction direction is “low,” “medium,” or “high,” and outputs the determination result to a display apparatus or the like.

Moreover, the storage unit 104 stores information of the gait disorder risk “low” when the abnormality degree a(W) of the whipping motion in the abduction direction for the right foot satisfies 0≤a(W)≤αth2, stores information of the gait disorder risk “medium” when the abnormality degree a(W) for the right foot satisfies αth2<a(W)≤βth2, and stores information of the gait disorder risk “high” when the abnormality degree a(W) for the right foot satisfies βth2<a(W). On the basis of this information, the risk determination unit 13 determines whether the gait disorder risk of the whipping motion of the right foot in the abduction direction is “low,” “medium,” or “high,” and outputs the determination result to a display apparatus or the like. Similarly, the storage unit 104 stores information of the gait disorder risk “low” when the abnormality degree a(W) of the whipping motion in the adduction direction for the right foot satisfies 0≤a(W)≤αth2′, stores information of the gait disorder risk “medium” when the abnormality degree a(W) for the right foot satisfies αth2′<a(W)≤βth2′, and stores information of the gait disorder risk “high” when the abnormality degree a(W) for the right foot satisfies βth2′<a(W). On the basis of this information, the risk determination unit 13 determines whether the gait disorder risk of the whipping motion of the right foot in the adduction direction is “low,” “medium,” or “high,” and outputs the determination result to a display apparatus or the like.

### Second Example Embodiment

FIG. 12 is a functional block diagram of a determination apparatus according to a second example embodiment.

In the above-described processing, the first sensor apparatus 2 calculates the feature amount of the whipping motion of the left foot, and the second sensor apparatus 3 calculates the feature amount of the whipping motion of the right foot. However, the first sensor apparatus 2 may transmit sensing information of the left foot to the determination apparatus 1, the second sensor apparatus 3 may transmit sensing information of the right foot to the determination apparatus 1, and the determination apparatus 1 may detect whipping motion determination periods for the left foot and the right foot, calculate feature amounts for the left foot and the right foot, and determine gait disorder risks for the left foot and the right foot on the basis of the sensing information of each foot.

In this case, as shown in FIG. 12, a determination apparatus 1 includes a control unit 11, a left foot information acquisition unit 121, a right foot information acquisition unit 122, a left foot determination period detection unit 131, a right foot determination period detection unit 132, a left foot feature amount calculation unit 141, a right foot feature amount calculation unit 142, a left foot risk determination unit 151, and a right foot risk determination unit 152.

Then, the left foot information acquisition unit 121 acquires sensing information of the left foot. The left foot determination period detection unit 131 detects a whipping motion determination period of the left foot in the same manner as the processing described above. The left foot feature amount calculation unit 141 calculates a feature amount of a whipping motion of the left foot in the same manner as the processing described above. The left foot risk determination unit 151 determines a gait disorder risk of the left foot in the same manner as the processing described above.

Similarly, the right foot information acquisition unit 122 acquires sensing information of the right foot. The right foot determination period detection unit 132 detects a whipping motion determination period of the right foot in the same manner as the processing described above. The right foot feature amount calculation unit 142 calculates a feature amount of a whipping motion of the right foot in the same manner as the processing described above. The right foot risk determination unit 152 determines a gait disorder risk of the right foot in the same manner as the processing described above.

### Third Example Embodiment

The risk determination unit 13 may determine a gait disorder risk on the basis of a comparison between a feature amount related to a user and past feature amounts related to the user. In this case, the determination apparatus 1 stores an average value μ and a standard deviation 6 of feature amounts W of past whipping motions of the user obtained from a normal distribution model, which is a statistical model for determining a gait disorder risk, in a storage unit 104 in advance. This statistical model is a statistical model created from the user's own past whipping motions. The risk determination unit 13 calculates abnormality degrees a(W) of feature amounts W of whipping motions for both feet in the same manner using the above-described Equation (6).

Then, the risk determination unit 13 determines whether the abnormality degree a(W) of the feature amount W of the whipping motion calculated for each of the both feet exceeds a threshold αth of the abnormality degree. The risk determination unit 13 determines that there is a risk for the left foot or the right foot if the abnormality degree a(W) of the feature amount W of the whipping motion of the left foot or the right foot exceeds the threshold αth of the abnormality degree, and determines that there is no risk for the left foot or the right foot if the abnormality degree a(W) of the feature amount W of the whipping motion of the left foot or the right foot does not exceed the threshold αth of the abnormality degree.

The risk determination unit 13 determines whether there is a risk or no risk for the left foot or the right foot as to the abnormality degree a(W) for each motion cycle of the walking motion, and when a predetermined ratio of the determination results of the abnormality degree a(W) for the left foot or the right foot for all the motion cycles generated in a predetermined period during the walking is at risk, the risk determination unit 13 outputs information that there is a risk for the left foot or the right foot to a display apparatus such as a liquid crystal screen. For example, when the determination apparatus 1 is a smartphone, the information that there is a risk for the left foot or the right foot is displayed on a liquid crystal screen of the smartphone. Thereby, the user can learn that he/she is performing walking motion having a risk for the left foot or the right foot in his/her own walking motion as compared with his/her own past walking motion.

### Fourth Example Embodiment

The risk determination unit 13 may determine a gait disorder risk on the basis of a comparison between a feature amount related to one foot of a user and a feature amount related to the other foot of the user. In this case, the determination apparatus 1 separately stores average values μ and standard deviations 6 of past feature amounts W of whipping motions for the left foot and right foot of the user obtained from a normal distribution model, which is a statistical model for determining a gait disorder risk, in the storage unit 104 in advance. This statistical model is each individual statistical model created from user's own past whipping motions for each of the left foot and right foot. The risk determination unit 13 calculates an abnormality degree a_{L}(W) of a feature amount W_{L} of a whipping motion of the left foot and an abnormality degree a_{R}(W) of a feature amount W_{R} of a whipping motion of the right foot in the same manner using the above-described Equation (6).

Then, the risk determination unit 13 determines whether the abnormality degree a_{L}(W) of the feature amount W_{L} of the whipping motion calculated for the left foot exceeds a threshold αth_{R} of the abnormality degree of the right foot. The risk determination unit 13 determines that there is no risk for the left foot if the abnormality degree a_{L}(W) of the feature amount W_{L} of the whipping motion calculated for the left foot does not exceed the threshold αth_{R} of the abnormality degree of the right foot. Similarly, the risk determination unit 13 determines that there is no risk for the right foot if the abnormality degree a_{R}(W) of the feature amount W_{R} of the whipping motion calculated for the right foot does not exceed a threshold αth_{L} of the abnormality degree of the left foot.

This processing is an example aspect of the processing of determining a gait disorder risk on the basis of the comparison between the feature amount of one foot of the user and the feature amount of the other foot of the user. With such processing, the gait disorder risk can be determined on the basis of a difference in whipping motions between the left foot and the right foot.

### Fifth Example Embodiment

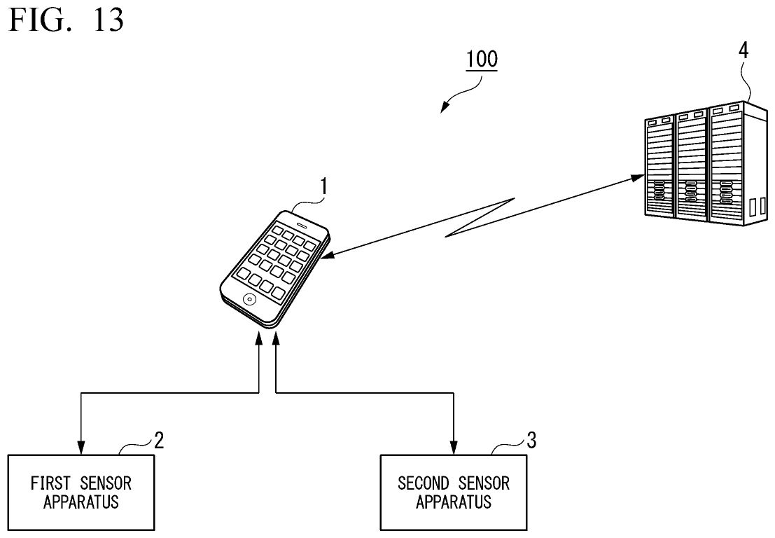

FIG. 13 is a diagram showing a schematic configuration of a gait disorder risk determination system according to a fifth example embodiment.

The gait disorder risk determination system 100 may further include a server apparatus 4, and the server apparatus 4 may perform part of the processing of the determination apparatus 1 described above. That is, the server apparatus 4 may perform at least one of the determination period detection processing, the feature amount calculation processing of the whipping motion, and the gait disorder risk determination processing explained for the determination apparatus 1 described above. In this case, the server apparatus 4 receives information for performing the processing via the determination apparatus 1 and returns a result of the processing to the determination apparatus 1. Then, the determination apparatus 1 outputs the result of the gait disorder risk determination on the basis of the information returned from the server apparatus 4.

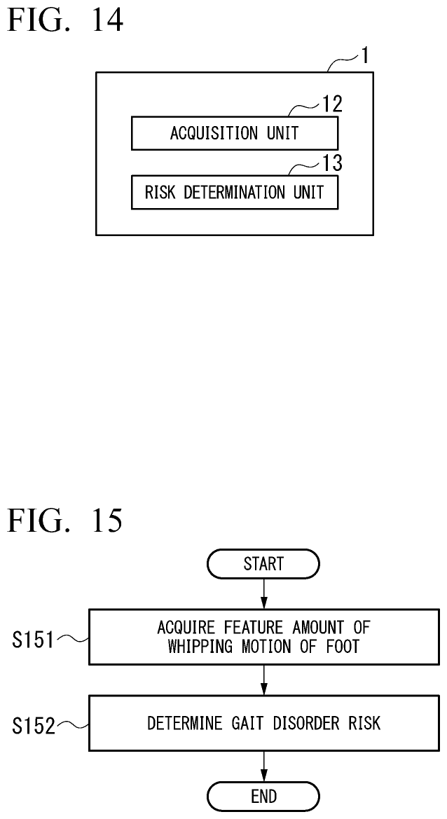

FIG. 14 is a diagram showing a minimum configuration of the determination apparatus 1.

FIG. 15 is a diagram showing a processing flow of the determination apparatus 1 with the minimum configuration.

The determination apparatus 1 includes at least an acquisition unit 12 and a risk determination unit 13.

The acquisition unit 12 acquires a feature amount of a whipping motion of a foot (step S151).

Moreover, the risk determination unit 13 determines a gait disorder risk on the basis of the feature amount of the whipping motion (step S152).

Each of the above-described apparatuses includes a computer system therein. A process of each processing described above is stored on a computer-readable recording medium in a form of a program, and the above-described processing is performed by the computer reading and executing the program. Here, the “computer-readable recording medium” refers to a magnetic disk, a magneto-optical disk, a compact disc (CD)-ROM, a digital versatile disc (DVD)-ROM, a semiconductor memory, or the like. Moreover, the computer program may be delivered to the computer via a communication link, and the computer to which the computer program has been delivered may execute the program.

Moreover, the above-described program may be a program for realizing some of the above-described functions. Furthermore, the above-described program may be a so-called differential file (differential program) which realizes the above-described functions in combination with a program already recorded on the computer system.

While the present invention has been particularly shown and described with reference to example embodiments thereof, the present invention is not limited to these example embodiments. It will be understood by those of ordinary skill in the art that various changes in form and details may be made therein without departing from the spirit and scope of the present invention as defined by the claims.

What is claimed is:

1. A determination apparatus comprising:
at least one memory configured to store instructions; and
at least one processor configured to execute the instructions to:
acquire a feature amount of a whipping motion of a foot; and
determine a gait disorder risk on the basis of the feature amount.

2. The determination apparatus according to claim 1, wherein the at least one processor is configured to calculate the feature amount on the basis of an acceleration or an angular velocity of the foot.

3. The determination apparatus according to claim 2, wherein the at least one processor is configured to calculate the feature amount in a whipping motion determination period from a pre-swing to an initial swing of any leg in a walking motion cycle of a person.

4. The determination apparatus according to claim 1, wherein the at least one processor is configured to determine the gait disorder risk on the basis of a comparison between the feature amount related to a user and a past feature amount related to the user.

5. The determination apparatus according to claim 1, wherein the at least one processor is configured to determine the gait disorder risk on the basis of a comparison between the feature amount related to one foot of a user and the feature amount related to the other foot of the user.

6. The determination apparatus according to claim 1, wherein the at least one processor is configured to determine the gait disorder risk on the basis of a comparison between the feature amount related to a user and a feature amount related to another user.

7. The determination apparatus according to claim 2, wherein
the acceleration or the angular velocity is measured by a sensor provided in a sole of a shoe attached to the foot, and
the feature amount is a rotation angle around a leg axis with a vicinity of an ankle of the foot as an origin.

8. A sensor apparatus provided in a shoe sole, the sensor apparatus comprising:
at least one memory configured to store instructions; and
at least one processor configured to execute the instructions to:
measure an acceleration or an angular velocity of a foot; and
calculate a feature amount of a whipping motion of the foot on the basis of the acceleration or the angular velocity.

9. A determination method comprising:
acquiring a feature amount of a whipping motion of a foot; and
determining a gait disorder risk on the basis of the feature amount.

10. A non-transitory computer-readable recording medium that records a program for causing a computer of a determination apparatus to execute the determination method according to claim 9.
